SEQUENCE LISTING (1 sequence)

SEQ ID NO: 1 - DNA, 1573 bp, Lactobacillus salivarius
tgggcgagtcgcatgctccggccgccatggcggccgcgggaattcgattacggttaccttgttacgacttcaccccaatcatctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccaacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcgtccccaccttcctccggtttgtcaccggcagtctcgccagagtgcccaacttaatgctggcaactgacaacaagggttgcgctcgttgcgggacttaacccaacatctcacgacacgagctgacgacagccatgcaccacctgtcactttgtccccgaagggaaaacctaatctcttaggtggtcaaaggatgtcaagacctggtaaggttcttcgcgttgcttcgaattaaaccacatgctccaccgctgtgcgggcccccgtcaattcctttgagtttcaaccttgcggtcgtactccccaggcggaatgcttattgcgttagctgcggcactgaagggcggaaaccctccaacacctagcattcatcgtttacggcgtggactaccagggtatctaatcctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactactccggttaagccgaaggctttcacatcagacttaaaagaccgcctgcgttccctttacgcccaataaatccggacaacgcttgccacctacgtattaccgcggctgctggcacgtagttagccgtgacttgctggttagataccgtcatcgaatgaacagttactctcactcgtgttcttctctaacaacagagttttacgatccgaagaccttcttcactcacgcggcgttgctccatcagacttgcgtccattgtggaagattccctactgctgcctcccgtaggagtttgggccgtgtctcagtcccaatgtggccgatcaacctctcagttcggctacgtatcatcaccttggtaggccgttaccccaccaactagttaatacgccgcgggtccatctaaaagcgatagcagaaccatctttcatctaagaatcatgcgatccttagagatatacggtattagcacctgtttccaagtgttatccccttcttttaggcaggttacccacgtgttactcacccgtccgccactcaacttcttacggtgaatgcaagcattcggtgtaagaaagtttcgttcgacttgcatgtattaggcacgccgccagcgttcgtcctgagccaggatcaaactct

(19) United States
(12) Patent Application Publication
(10) Pub. No.: US 2011/0318281 A1
(43) Pub. Date: Dec. 29, 2011

(54) Lactobacillus Salivarius SG-M6 for Improving Oral Bacterial Groups and Health Care Compositions Thereof

(75) Inventors: Chu Yang Hsieh, Tainan (TW); Fang Yun Tai, Tainan (TW); Chi Chiang Yang, Taichung City (TW)

(73) Assignee: SYNGEN BIOTECH CO., LTD., Hsin-Ying Tainan (TW)

(21) Appl. No.: 12/900,952

(22) Filed: Oct. 8, 2010

(30) Foreign Application Priority Data
Jun. 23, 2010 (TW) 099120476

(51) Int. Cl. A61K 35/74; A61P 1/02; C12N 1/20

(52) U.S. Cl. 424/50; 435/252.9

(57) ABSTRACT

This invention provides to a strain of Lactobacillus salivarius SG-M6 with deposit number of CGMCC NO. 3505 and a fermentation product thereof. The present invention also provides a health composition for improving oral bacteria population, which comprises the Lactobacillus salivarius SG-M6 or the fermentation product thereof. The health care composition can be applied to prevention or treatment of oral bacterial diseases.

### FIELD OF THE INVENTION

The present invention relates to a Lactobacillus salivarius strain SG-M6 for improving oral bacterial groups, inhibiting bacterial growth in oral and a fermentation product thereof.

### BACKGROUND OF THE INVENTION

Normal human mouth contains lots of bacteria, fungi, and even viruses, where bacteria are the major population. There are 100 million bacteria in one milliliter of saliva, and the whole mouth contains more than 600 kinds of bacteria. However, not all of these bacteria are pathogens. Certain probiotics are also present. These bacteria maintain a relative balance and are non-pathogenic under normal condition. Under the situation of low immunity or resistance, oral environmental changes, medication, or systematic disease, over growth of pathogens result in oral diseases, as minor as bad breath, dental plaque, gingivitis or as severe as cavity, periodontal disease and even bacteremia caused by massive bacteria reproduction after blood vessels infection.

According to the Annual National Health Insurance statistic analysis by Taiwan Dental Association, 90% of adults in Taiwan area suffering periodontal diseases demonstrated the importance of oral hygiene.

There are two kinds of periodontal diseases, i.e., gingivitis and periodontitis. The major symptoms of gingivitis are gum bleeding, swelling, redness and etc. Periodontitis indicates the status of destroyed alveolar bone which supports gum tissue and teeth.

One of the periodontal disease symptoms is deepen grove between teeth and gum resulting in periodontal pocket. The periodontal pocket of people with healthy teeth is around 1-2 mm, which is 3-4 mm of mild periodontal disease patients, 4-6 mm of moderate patients, and over 6 mm of severe patients. Along with deeper periodontal pocket, the gum is getting shorter. The appearance of longer or mobile teeth could be the warning sign of severe periodontal diseases. Gingivitis and periodontitis are caused by periodontal bacterial infection. The representative bacterium is Porphyromonas gingivalis.

Glycoprotein in saliva forms a film on the teeth surface which enables bacteria attachment. After bacteria utilizing the sugar of food, the film is getting bigger and thicker resulting in dental plaque. Moreover, if the dental plaque is formed in periodontal pocket, periodontal bacteria will reproduce massively in the dental plaque. Due to anaerobic characteristics of periodontal bacteria, the oxygen unreachable periodontal pocket is their most optimal growth condition. Dental plaque is also the incubator of Streptococcus mutans. More than 90% of adults carry this type of bacteria in their mouth, which is also the major bacteria strain causing cavity. When acidic substances generated from sugar degradation by Streptococcus mutans erode enamel and dentin, it will cause cavity.

Periodontal bacteria invading gum will cause immunological reactions. Periodontal bacteria secret enzymes dissolving gum cell and invading the internal side of the gum. With minimal level of periodontal bacteria, the invasion can be stopped. As long as massive reproduction occurs, the reproduction can not be inhibited.

There are some present methods to prevent the formation of cavity or periodontal diseases, such as applying anti-adhesives to avoid bacteria attachment on teeth surface, and to minimize dental plaque formation, and bacteria erosion of dentin (R.O.C. patent application number 094144377); applying anti-bacterial reagent to inhibit bacteria growth (U.S. Pat. No. 5,368,845; WO 92/14475); or widely used fluoride to reduce the solubility of enamel against acidic substances for cavity prevention.

### BRIEF DESCRIPTION OF THE DRAWINGS

FIG. 1A and FIG. 1B shows nucleotide sequence comparison of SEQ ID NO: 1 of the present invention and known Lactobacillus salivarius (bacterial strain ATCC11741 in US patent publication No. 2002/0094328), using NCBI blast program. Query sequence is ATCC11741, and Subject sequence is SEQ ID NO: 1 of the present invention.

FIG. 2 shows liver tissue section of Lactobacillus salivarius SG-M6 treatment group (tLSP15) (200×).

FIG. 3 shows kidney tissue section of Lactobacillus salivarius SG-M6 treatment group (tLSP15) (200×).

FIG. 4 shows liver tissue section of control group with tetracycline treatment (tTC) (200×).

FIG. 5 shows kidney tissue section of control group with tetracycline treatment (tTC) (200×).

FIG. 6 shows liver tissue section of control group with no treatment (distilled water) (tMT) (200×).

FIG. 7 shows kidney tissue section of control group with no treatment (distilled water) (tMT) (200×).

FIG. 8 shows liver tissue section of Lactobacillus salivarius SG-M6 prevention group (pLSP15) (200×).

FIG. 9 shows kidney tissue section of Lactobacillus salivarius SG-M6 prevention group (pLSP15) (200×).

FIG. 10 shows liver tissue section of control group with tetracycline prevention treatment (pTC) (200×).

FIG. 11 shows kidney tissue section of control group with tetracycline prevention treatment (pTC) (200×).

FIG. 12 shows liver tissue section of control group with no prevention treatment (distilled water) (pMT) (200×).

FIG. 13 shows kidney tissue section of control group with no prevention treatment (distilled water) (pMT) (200×).

### SUMMARY OF THE INVENTION

This invention discloses a strain of Lactobacillus salivarius SG-M6 with deposit number of CGMCC NO. 3505 and a fermentation products thereof.

The present invention also discloses a health composition for improving oral bacteria population, which comprises the Lactobacillus salivarius SG-M6 or the fermentation product thereof.

### DETAILED DESCRIPTION OF THE INVENTION

The present invention provides a strain of Lactobacillus salivarius SG-M6 (deposit number CGMCC NO. 3505). The present invention further reveals that the Lactobacillus salivarius SG-M6 includes codons as nucleotide sequences shown in SEQ ID NO: 1. The present invention also reveals the fermentation product of medium cultured Lactobacillus salivarius SG-M6.

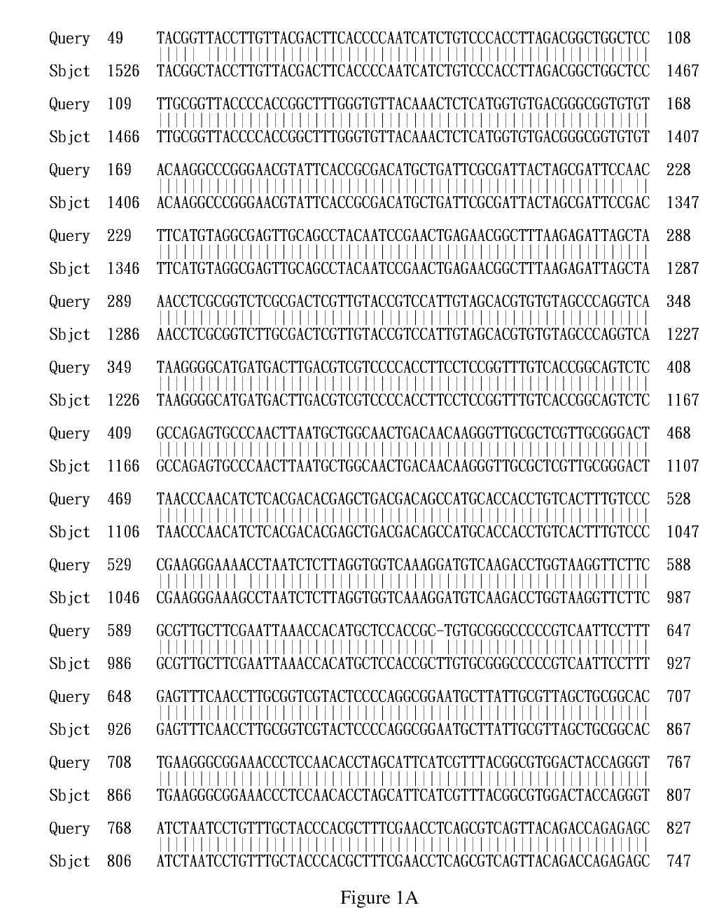

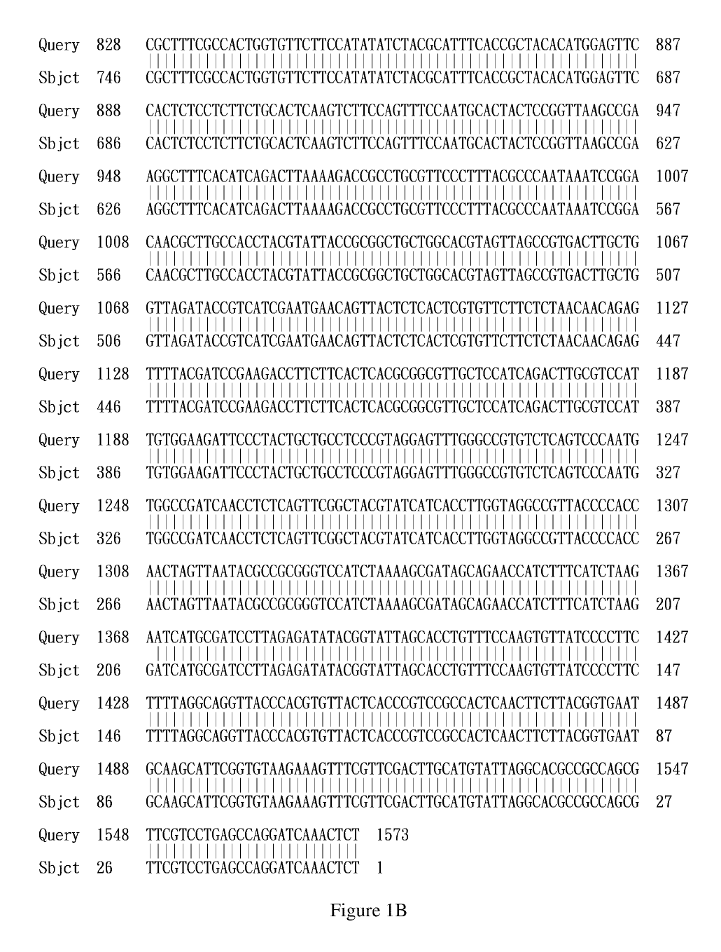

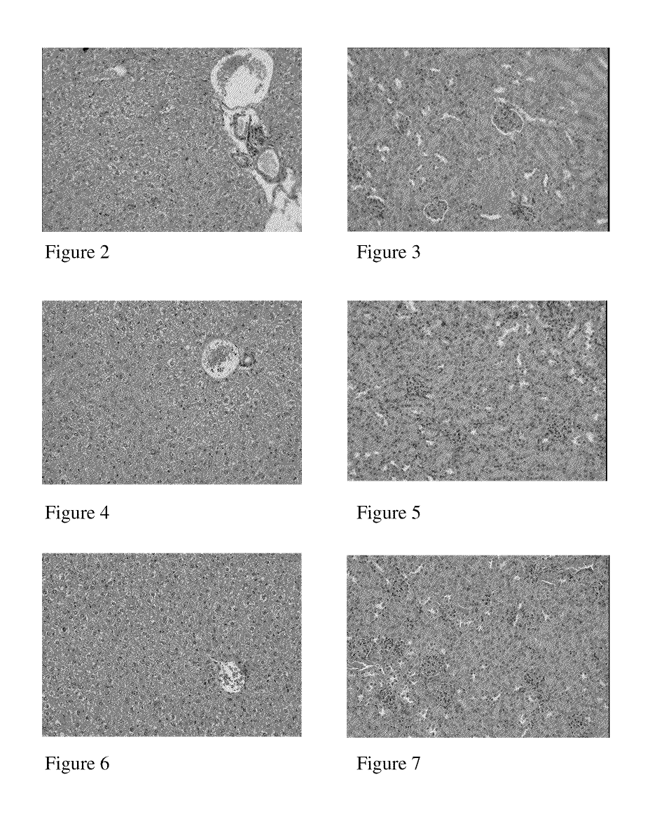

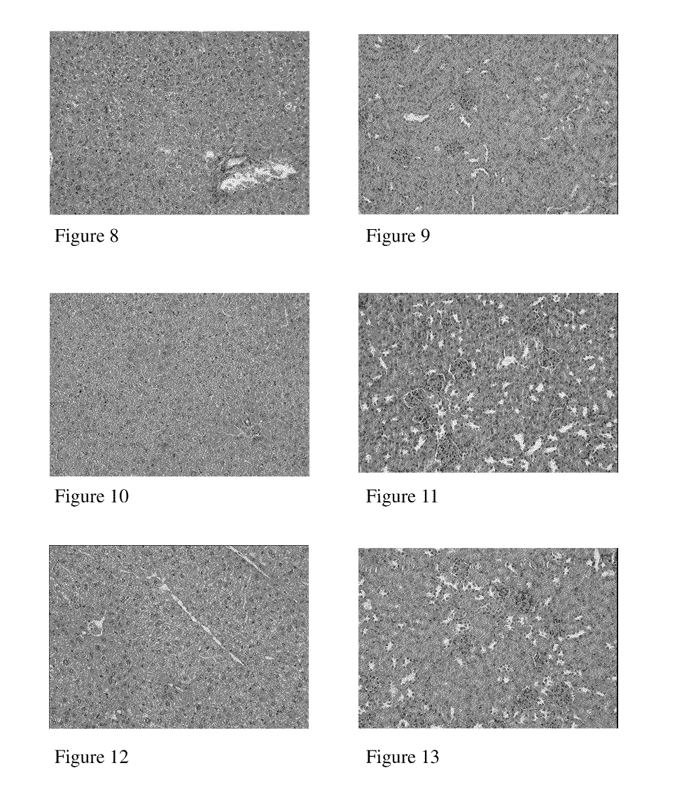

The present invention also provides a health care composition for improving oral bacteria population. An application of said Lactobacillus salivarius SG-M6 or its fermentation product on oral hygiene can effectively inhibit oral periodontal bacteria growth. SEQ ID NO: 1 of the present invention has significant difference as compared to the genetic material of known Lactobacillus salivarius (shown in FIG. 1). The API 50 CHL comparison identifying by API BioMerieux Research laboratory, La Balme Les Grottes, Montalien, Jeraeh, France, revealed significant difference of Lactobacillus salivarius SG-M6 of the present invention against EU patent 1312667 revealed strain WB 21 (deposit number FERM P-17991), US patent publication number 2007/0071737 revealed strain TI2711 (deposit number FERM BP-7974) and EU patent 0154549 disclosed strain AD0001 (deposit number FERM P-7537) as shown in Table 1.

TABLE 1
API 50 CHL comparison of Lactobacillus salivarius SG-M6 of the present invention
and EU patent 1312667 revealed strain WB21 (deposit number FERM P-17991), US patent
publication number 2007/0071737 revealed strain TI2711 (deposit number FERM BP-7974)
and EU patent 0154549 revealed strain AD0001 (deposit number FERM P-7537).

| | | Lactobacillus | EP1312667 | US2007/0071737 | EP0154549 |
|---|---|---|---|---|---|
| | | Salivarius of the | (WB21) | (TI2711) | (AD0001) |
| | | present invention | (FERM | (FERM | (FERM |
| API 50 CHL | Sugar group | (SG-M6) | P-17991) | BP-7974) | P-7537) |
| 0 | Control | − | | | |
| 1 | Glycerol | − | | | |
| 2 | Erythritol | − | | | |
| 3 | D-Arabinose | − | − | − | − |
| 4 | L-Arabinose | − | | | |
| 5 | D-Ribose | + | − | − | − |
| 6 | D-Xylose | − | − | − | − |
| 7 | L-Xylose | − | | | |
| 8 | Adonitol | + | | | |
| 9 | β-Methyl-xyloside | − | | | |
| 10 | Galactose | + | + | + | + |
| 11 | D-Glucose | + | + | + | + |
| 12 | D-Fructose | + | + | + | |
| 13 | D-Mannose | + | + | + | + |
| 14 | L-Sorbose | − | | | |
| 15 | Rhamnose | + | + | + | |
| 16 | Dulcitol | − | | | |
| 17 | Inositol | − | | | |
| 18 | Mannitol | + | + | + | + |
| 19 | Sorbitol | + | + | + | + |
| 20 | α-Methyl-D-mannoside | − | | | |
| 21 | α-Methyl-D-glucoside | − | | | |
| 22 | N-Acetyl glucosamine | + | | | |
| 23 | Amygdaline | − | − | − | − |
| 24 | Arbutine | − | | | |
| 25 | Esculine | + | − | − | + |
| 26 | Salicine | + | − | − | + |
| 27 | Cellobiose | − | − | − | − |
| 28 | Maltose | + | + | + | + |
| 29 | Lactose | + | + | + | + |
| 30 | Melibiose | + | + | + | + |
| 31 | Saccharose | + | + | + | + |
| 32 | Trehalose | + | + | + | + |
| 33 | Inuline | − | | | |
| 34 | Melezitose | − | − | − | − |
| 35 | D-Raffinose | + | + | + | + |
| 36 | Amidon | − | | | |
| 37 | Glycogen | − | | | |
| 38 | Xylitol | − | | | |
| 39 | β-Gentiobiose | − | | | |
| 40 | D-Turanose | − | | | |
| 41 | D-Lyxose | − | | | |
| 42 | D-Tagatose | − | | | |
| 43 | D-Fucose | − | | | |
| 44 | L-Fucose | − | | | |
| 45 | D-Arabitol | − | | | |
| 46 | L-Arabitol | − | | | |
| 47 | Gluconate | − | | | |
| 48 | 2 keto-gluconate | − | | | |
| 49 | 5 keto-gluconate | − | | | |

Lactobacillus salivarius SG-M6 fermentation product in the present invention is the product after fermentation of Lactobacillus salivarius SG-M6 via culture medium containing appropriate ingredients for Lactobacillus salivarius SG-M6 growth. The major ingredients of said medium include glucose, peptone, meat extract, yeast extract, salt and etc. The bacteria inhibition activity of Lactobacillus salivarius SG-M6 fermentation product is not affected by dialyzing; for example, dialyze by Spectra/Por® dialysis membrane (MWCO:3500).

The periodontal bacteria are affected by health care composition to the level of seizing growth or even death. Based on the characteristics of inhibiting oral bacteria growth such as Streptococcus mutans, Streptococcus sanguis, Porphyromonas gingivalis and Actinomyces viscosus, the composition of the present invention could be applied to prevent or treat oral bacterial diseases as follows:
1. Porphyromonas gingivalis related diseases: periodontal disease, osteoporosis, cavernous sinus thrombophlebitis, periodontitis, cardiovascular disease, infective endocarditis, diabetes, respiratory disease, atherosclerosis, coronary heart disease, stroke, and rheumatoid arthritis.
2. Streptococcus sanguis related diseases: cavity, infective endocarditis, acute septic arthritis, and cardiovascular disease.
3. Streptococcus mutans related diseases: cavity and infective endocarditis.
4. Actinomyces viscosus related diseases: periodontitis.

The present invention discloses a health care composition, wherein the live Lactobacillus salivarius SG-M6 and its fermentation product can inhibit bacteria growth. Therefore, Lactobacillus salivarius SG-M6 and its fermentation product can be applied to directly spread on the oral cavity. It is also possible to freeze or dry bacteria of the present invention without compromising the reconstitute activity of bacteria strain or its fermentation product in the oral cavity, and in the form of tablet, spray, solution, or additive of food, pharmaceutical or oral hygiene for application. Any method which does not compromise the bacterial inhibition ability of the health care composition can be the applicable method for the present invention.

The subject in the present invention is mammal. In a preferred embodiment, the subject is human.

### Example

The invention is further described in the following examples, which are non-limiting the scope of the present invention and are merely representative of the preferred embodiments of the present invention.

The present invention disclosed a health care composition of preventing or treating oral bacterial diseases wherein the active ingredient included fermentation product of Lactobacillus salivarius SG-M6. In vitro or in vivo study in the present invention demonstrated the therapeutic effects of fermentation product of said bacteria against periodontal diseases.

### 1. In Vitro Study

Lactobacillus salivarius SG-M6 were cultured in liquid medium. MRS medium was used in this embodiment. The bacteria were cultured at 30-37 for 15-24 hours. The fermentation broth was concentrated into 30-fold of original concentration, dialyzed by Spectra/Por®Dialysis Membrane (MWCO:3500, Spectrum Laboratories Inc, CA) for 48 hours, and diluted into various concentrations according to experimental requirements. Disc agar diffusion test, broth dilution method, and co-cultured test of Lactobacillus salivarius SG-M6 with Streptococcus mutans (ATCC 25175), Streptococcus sanguis (ATCC 49295), Porphyromonas gingivalis (ATCC 33277), and Actinomyces viscosus (ATCC 15987) were separately performed to understand the inhibition of pathogen growth. Above four pathogens strains were purchased from Bioresource Collection and Research Center of Food Industry Research and Development Institute in Taiwan or ATCC.

### (1) Disc Agar Diffusion Test

Streptococcus mutans, Streptococcus sanguis, Porphyromonas gingivalis and Actinomyces viscosus were cultured to 0.5 McFarland turbidity. Dip bacteria culture with sterile cotton swab for 3 seconds, and each bacteria was smeared on agar plates in three directions to evenly distribute the inoculum. After dialyzing the concentrate of Lactobacillus salivarius SG-M6, adjust the dialyzed concentrate to 15-fold concentrate (LFP15), 10-fold concentrate (LFP10), 5-fold concentrate (LFP5), 4-fold concentrate (LFP4), 2-fold concentrate (LFP2) and 1-fold (LFP1) (unconcentrated fermentation broth). 6 mm diameter autoclaved paper disk was soaked in different concentrations of Lactobacillus salivarius SG-M6 fermentation product or live Lactobacillus salivarius SG-M6 suspension (LSB) for 3 seconds. Paper discs were placed on the agar plates, the plates were incubated in a 37 anaerobic incubator for 24 hours, and the sizes of inhibition zones were measured.

### (2) Broth Dilution Method

After dialyzing the 30-fold fermentation product of Lactobacillus salivarius SG-M6, the dialyzed fermentation product was serial diluted into 15-fold concentrate (LFP15), 10-fold concentrate (LFP10), 5-fold concentrate (LFP5), 4-fold concentrate (LFP4), 2-fold concentrate (LFP2), and 1-fold (LFP1). Each of Streptococcus mutans, Streptococcus sanguis, Porphyromonas gingivalis and Actinomyces viscosus were cultured in sterile BHI broth to 0.5 McFarland turbidity. 50 μL of bacteria suspension was added into each tube and cultured in a 37 anaerobic incubator for 48 hours. Plate counts were performed to calculate bacteria colony number for estimating bacterial inhibition concentrations of samples.

(3) Co-Culture Test of Lactobacillus salivarius SG-M6 and Pathogens

Live Lactobacillus salivarius SG-M6 was placed with Streptococcus mutans, Streptococcus sanguis or Porphyromonas gingivalis into test tubes, and co-cultured at 37. Samples were taken at different time points. Plate counts were preformed to calculate bacteria colony number to investigate pathogen growth inhibition of samples.

### 2. In Vivo Study

This trial included treatment groups and control groups. The treatment groups of Lactobacillus salivarius SG-M6 were divided into 6 groups as follows: 6 groups of dialyzed fermentation product concentrate of Lactobacillus salivarius SG-M6 included 15-fold, 10-fold, 5-fold, 4-fold, 2-fold, and 1-fold (unconcentrated fermentation broth). Two control groups included the treatment of 0.267 mg/mL of tetracycline and distilled water.

### (1) Evaluation of Preventive Effect for Animal Periodontal Disease

Twelve of 8-week-old female Balb/c mice were selected for each group (Table 2). The mice were administered with various concentrations of Lactobacillus salivarius SG-M6 fermentation product, 1 mL of 0.267 mg/mL tetracyccline or distilled water. Their mandibular anterior teeth were tied with a ligature wire, and inoculated periodontal pathogen Streptococcus mutans in gum tissue as an animal model of artificially induced periodontal disease. Their pathological symptoms observed and recorded until sacrificing all animals. Until the control group (feeding with distilled water, mock-treated group) showing gum swelling and dental plaque appearance diagnosed by chief dentists, the ligature wire was removed and inoculation of dental pathogen was stopped. At day 4, 8, 12, and 16 after wire removal, mice periodontal pocket depth was examined, 3 mice from each group were killed for blood sampling, and samples were collected for histological analysis.

TABLE 2
Prevention groups of animal study (Note: The tetracycline
dose of human is 1000 mg/75 kg body weight/day. The estimated
mice dose is about 0.267 mg/20 g body weight/day).

| | | | Group |
|---|---|---|---|
| | Applied dose (/mL) | | numbers |
| pLFP1 | Lactobacillus | 1 X | 8 |
| pLFP2 | salivarius SG-M6 | 2 X concentrate | |
| pLFP4 | fermentation product | 4 X concentrate | |
| pLSP5 | | 5 X concentrate | |
| pLSP10 | | 10 X concentrate | |
| pLSP15 | | 15 X concentrate | |
| pTC | Tetracycline | 0.267 mg | |
| pMT | Distilled water (mock | | |
| | treatment) | | |

### (2) Evaluation of Therapeutic Effect for Animal Periodontal Disease

Twelve of 8-week-old female Balb/c mice were selected for each group (Table 3). Until the control group showing gum swelling and dental plaque appearance, their mandibular anterior teeth were tied with the ligature wire, and inoculated periodontal pathogen Streptococcus mutans in gum tissue as an animal model of artificially induced periodontal disease. After diagnosis of diseased tissue by chief dentists, they were administered with different concentrations of Lactobacillus salivarius SG-M6 fermentation product, live Lactobacillus salivarius SG-M6, 1 mL of 0.267 mg/mL tetracycline or distilled water every day. The pathological symptoms were recorded. Wire was removed at the beginning of the treatment and periodontal pathogen inoculation was terminated. At day 4, 8, 12, and 16 after wire removal, mice periodontal pocket depth was examined. 3 mice from each group were killed for blood sampling and samples were collected for histological analysis.

TABLE 3
Animal study treatment group

| | | | Group |
|---|---|---|---|
| | Applied dose (per mL) | | numbers |
| tLSP1 | Fermentation product of | 1 X | 8 |
| tLSP2 | Lactobacillus salivarius | 2 X concentrate | |
| tLSP4 | SG-M6 | 4 X concentrate | |
| tLSP5 | | 5 X concentrate | |
| tLSP10 | | 10 X concentrate | |
| tLSP15 | | 15 X concentrate | |
| tTC | Tetracycline | 0.267 mg | |
| tMT | Distilled water (mock | | |
| | treatment) | | |

### (3) Improvement Percentage of Periodontal Pocket Depth

Control group was used as a standard to examine individuals in each group by Mann-Whitney test. Subjects with significant difference were defined as improving individuals. (number of improving Subject/number of total subject in group)×100%=improvement percentage of periodontal pocket depth.

### (4) Clinical Pathological and Blood Biochemical-Analysis

The mice were killed. Blood samples from carotid artery were centrifuged at 3000 rpm for 10 minutes at 4. Serum in the supernatant was taken, its ALT, AST, Creatinine, BUN biochemical indicator were analysis with an automated biochemical analyzer. important target organs such as liver and kidney were collected and fixed them with 10% formalin solution. After making parafilm section, H.E. staining was performed for pathological observation.

### 3. Statistic Analysis

Experimental data were shown as mean±S.D. The statistical variation of treatment group and control group of mice periodontal pocket were performed by One-Way ANOVA and Dunnett multiple post hoc comparison in longitudinal study, and by One-Way ANOVA and LSD multiple post hoc comparison in cross-sectional study. Besides, Mann-Whitney test was used for examining the number of periodontal pocket improving individuals. The p value of statistical significance level was 0.05.

### Results

### 1. In Vitro Study

### (1) Disc Agar Diffusion Test

Table 4 showed the inhibition effect of disc agar diffusion test. The diameter of sterile paper disc was 6 mm. The positive bacterial inhibition effect was determined by bacterial inhibition zone over 6 mm in diameter. Otherwise, it was defined as negative bacterial inhibition effect. Cultural condition was under the temperature of 37 for 24 hours. The paper disc was used in control group containing tetracycline. The inhibition zone values were shown as mean±S.D. (n=3).

TABLE 4
Bacterial inhibition effect of disc agar diffusion test

| | Actino- | Porphy- | Strepto- | Strepto- |
|---|---|---|---|---|
| | myces | romonas | coccus | coccus |
| | viscosus | gingivalis | mutans | sanguis |
| LSP1 | — | — | — | — |
| LSP2 | 10 ± 1.0 | 12 ± 1.0 | 9 ± 1.0 | 9 ± 1.0 |
| LSP4 | 13 ± 1.0 | 14 ± 1.0 | 13 ± 1.0 | 16 ± 1.0 |
| LSP5 | 15 ± 1.0 | 16 ± 1.0 | 15 ± 1.0 | 18 ± 1.0 |
| LSP10 | 23.2 ± 1.2 | 23 ± 1.0 | 21 ± 1.2 | 22 ± 1.0 |
| LSP15 | 31.1 ± 1.1 | 32 ± 1.1 | 29 ± 1.0 | 28 ± 1.4 |
| LSB | 14 ± 1.0 | 9 ± 0.5 | 14 ± 1.0 | 14 ± 1.0 |
| Tetracycline | 48.2 ± 2.4 | 33.4 ± 2.2 | 29.7 ± 2.0 | 25.9 ± 1.2 |

Table 4 showed that over 2-fold concentrate of Lactobacillus salivarius SG-M6 fermentation product and live Lactobacillus salivarius produced inhibition zone against these four pathogens, such as Actinomyces viscosus, Porphyromonas gingivalis, Streptococcus mutans, and Streptococcus sanguis. The denser the concentrate was presented, the larger the inhibition zone was produced which formed dose dependent effect.

### (2) Broth Dilution Method

TABLE 5
The minimal concentration of bacteria inhibition and inhibition percentage (100% −
(experimental group ÷ control group) × 100%).

| | Actinomyces | | Porphyromonas | | Streptococcus | | Streptococcus | |
|---|---|---|---|---|---|---|---|---|
| | viscosus | | gingivalis | | mutans | | sanguis | |
| | CFU/mL | Inhibitory | CFU/mL | Inhibitory | CFU/mL | Inhibitory | CFU/mL | Inhibitory |
| LSP15 | 4.78 × 10⁹ | 76.9% | 5.79 × 10⁸ | 93.4% | 8.64 × 10⁹ | 75.1% | 2.43 × 10¹⁰ | 65.9% |
| LSP10 | 5.63 × 10⁹ | 72.8% | 9.79 × 10⁸ | 88.9% | 1.09 × 10¹⁰ | 68.5% | 3.47 × 10¹⁰ | 51.2% |
| LSP5 | 8.29 × 10⁹ | 60.0% | 2.03 × 10⁹ | 77.0% | 2.09 × 10¹⁰ | 39.5% | 3.95 × 10¹⁰ | 44.5% |
| LSP4 | 1.15 × 10¹⁰ | 44.5% | 2.41 × 10⁹ | 72.7% | 2.39 × 10¹⁰ | 30.9% | 4.11 × 10¹⁰ | 42.2% |
| LSP2 | 1.43 × 10¹⁰ | 30.9% | 3.07 × 10⁹ | 65.2% | 3.07 × 10¹⁰ | 11.3% | 4.32 × 10¹⁰ | 39.3% |
| LSP1 | 2.09 × 10¹⁰ | 0% | 7.76 × 10⁹ | 11.9% | 3.10 × 10¹⁰ | 10.4% | 5.50 × 10¹⁰ | 22.6% |
| Control | 2.07 × 10¹⁰ | — | 8.81 × 10⁹ | — | 3.46 × 10¹⁰ | — | 7.11 × 10¹⁰ | — |

Table 5 showed different inhibition effects of Lactobacillus salivarius SG-M 6 fermentation product after treating periodontal pathogens 48 hours. In general, high concentration of Lactobacillus salivarius SG-M6 produced better inhibition effect. For periodontal pathogens, Lactobacillus salivarius SG-M6 fermentation product showed the most significant inhibition effect on Porphyromonas gingivalis in a dose-dependent manner. The second significant inhibition effect was against Actinomyces viscosus. Lactobacillus salivarius of over 2-fold concentrate showed inhibition effect against all these four pathogens.

(3) Co-Culture Test of Lactobacillus salivarius SG-M6 Fermentation Product or Live Lactobacillus salivarius and Pathogens

TABLE 6
Inhibition of pathogen growth by co-culture of Lactobacillus salivarius
SG-M6 fermentation product (LS) and Streptococcus mutans

| | Streptococcus mutans | | |
|---|---|---|---|
| Culture time | LS (2 X | LS(2.5 X | |
| (hour) | concentrate) | concentrate) | Control |
| 0 | 5.0 × 10⁷ | 5.0 × 10⁷ | 5.0 × 10⁷ |
| 1 | 1.5 × 10⁷ | 1.5 × 10⁷ | 6.0 × 10⁷ |
| 2 | 5.4 × 10⁶ | 3.1 × 10⁶ | 1.0 × 10⁸ |
| 3 | 3.1 × 10⁶ | 1.1 × 10⁴ | 1.5 × 10⁸ |
| 4 | 4.4 × 10⁵ | 0 | 6.0 × 10⁸ |
| 6 | 1.0 × 10³ | 0 | 1.0 × 10⁹ |
| 8 | 0 | 0 | 1.5 × 10⁹ |

Table 6 showed that after co-culture of 2-fold concentrate of Lactobacillus salivarius SG-M6 fermentation product and Streptococcus mutans for 8 hours, there were no Streptococcus mutans presented. There were no Streptococcus mutans presented after 4 hours co-culture of 2.5-fold concentrate and Streptococcus mutans. It demonstrated its good growth inhibition effect against Streptococcus mutans under low concentration of Lactobacillus salivarius SG-M6 fermentation product for short cultural time.

TABLE 7
Inhibition of pathogen growth by co-culture Lactobacillus salivarius
SG-M6 fermentation product (LS) and Streptococcus sanguis

| | Streptococcus sanguis | | |
|---|---|---|---|
| Culture time | LS (2 X | LS (2.5 X | |
| (hour) | concentrate) | concentrate) | Control |
| 0 | 5.0 × 10⁷ | 5.0 × 10⁷ | 5.0 × 10⁷ |
| 1 | 4.9 × 10⁵ | 8.0 × 10³ | 4.0 × 10⁷ |
| 2 | 6.9 × 10³ | 5.4 × 10² | 5.7 × 10⁷ |
| 3 | 1.2 × 10³ | 1.0 × 10² | 6.4 × 10⁷ |
| 4 | 3.4 × 10² | 0 | 8.1 × 10⁷ |
| 6 | 0 | 0 | 9.2 × 10⁷ |
| 8 | 0 | 0 | 1.0 × 10⁸ |

Table 7 showed that after co-culture of 2-fold concentrate of Lactobacillus salivarius SG-M6 and Streptococcus sanguis for 10 hours, there was no Streptococcus sanguis presented. There were no Streptococcus sanguis presented after 4 hours co-culture of 2.5-fold concentrate and Streptococcus sanguis. It demonstrated its good growth inhibition effect under low concentration of Lactobacillus salivarius SG-M6 fermentation product for short cultural time.

TABLE 8
Inhibition of pathogen growth by co-culture of Lactobacillus salivarius
SG-M6 fermentation product and Porphyromonas gingivalis

| | Porphyromonas gingivialis | | |
|---|---|---|---|
| Culture time | LS (2 X | LS (2.5 X | |
| (hour) | concentrate) | concentrate) | Control |
| 0 | 5.0 × 10⁷ | 5.0 × 10⁷ | 5.0 × 10⁷ |
| 1 | 3.1 × 10⁷ | 2.9 × 10⁶ | 5.3 × 10⁷ |
| 2 | 2.4 × 10⁶ | 8.5 × 10⁴ | 6.3 × 10⁷ |
| 3 | 8.4 × 10⁴ | 4.9 × 10³ | 8.3 × 10⁷ |
| 4 | 1.5 × 10³ | 6.4 × 10¹ | 1.3 × 10⁸ |
| 6 | 0 | 0 | 2.2 × 10⁸ |
| 8 | 0 | 0 | 4.1 × 10⁸ |

Table 8 showed that after co-culture of 2- or 2.5-fold concentrate of Lactobacillus salivarius SG-M6 fermentation product and Porphyromonas gingivalis for 6 hours, there was no Porphyromonas gingivalis presented. It demonstrated its good growth inhibition effect against Porphyromonas gingivalis under low concentration of Lactobacillus salivarius SG-M6 fermentation product for short cultural time.

TABLE 9
Inhibition of pathogen growth by co-culture of live Lactobacillus
salivarius SG-M6 (LSB) and Streptococcus mutans

| Culture time | Streptococcus mutans | | |
|---|---|---|---|
| (hour) | LSB(10⁷) | LSB(10⁸) | Control |
| 0 | 5.0 × 10⁷ | 5.0 × 10⁷ | 5.2 × 10⁷ |
| 4 | 7.4 × 10⁶ | 7.8 × 10⁶ | 4.9 × 10⁸ |
| 8 | 5.9 × 10⁵ | 6.1 × 10⁵ | 1.5 × 10⁹ |
| 24 | 26 | 17 | 1.7 × 10⁹ |
| 32 | 0 | 0 | 2.5 × 10⁷ |

Table 9 showed the co-culture of live Lactobacillus salivarius SG-M6 and Streptococcus mutans. There was no Streptococcus mutans presented in low bacteria counts group (10⁷) and median bacteria counts group (10⁸) for 32 hours co-culture. It demonstrated its growth inhibition effect against Streptococcus mutans.

### 2. In Vivo Study

### (1) Evaluation of Prevention Effect for Animal Periodontal Disease

TABLE 10
Comparison of the mice periodontal pocket depth (mm) in Lactobacillus salivarius
SG-M6 prevention group (pLF) with control group (pMT) at different
time points (One-Way ANOVA and Dunnett multiple post hoc comparison;
*p < 0.05; **p < 0.01).

| | Day 4 | Day 8 | Day 12 | Day 16 |
|---|---|---|---|---|
| pLSP1 | 1.33 ± 0.58 | 1.17 ± 0.76 | 0.67 ± 0.29** | 0.75 ± 0.35** |
| pLSP2 | 0.50 ± 0.00** | 0.83 ± 0.29** | 1.67 ± 1.15 | 1.50 ± 0.71 |
| pLSP4 | 1.23 ± 1.54 | 0.63 ± 0.75** | 0.40 ± 0.17** | 0.35 ± 0.21** |
| pLSP5 | 0.90 ± 0.52** | 0.59 ± 0.26** | 0.50 ± 0.00** | 0.20 ± 0.00** |
| pLSP10 | 0.96 ± 0.50** | 0.83 ± 0.50** | 0.40 ± 0.15** | 0.30 ± 0.17** |
| pLSP15 | 0.73 ± 0.30** | 0.47 ± 0.10** | 0.40 ± 0.15** | 0.40 ± 0.17** |
| pTC | 0.84 ± 0.50** | 0.70 ± 0.34** | 0.56 ± 0.26** | 0.49 ± 0.25** |
| pMT | 2.22 ± 1.07 | 2.16 ± 1.25 | 2.75 ± 1.32 | 2.21 ± 0.64 |

Effects of different concentrations of Lactobacillus salivarius SG-M6 fermentation product on periodontal pocket depth in prevention groups were evaluated. By comparing with control group (pMT) treated with distilled water at each time point, the loss of periodontal pocket depth showed significant difference at day 4 to 16 and groups of pLSP15, pLSP10 and pLSP5. Significant difference was also observed in pLSP4 group at all time points, except for day 4. Significant difference was observed in pLSP1 group at day 12 and day 16. Significant difference was observed in prevention control group (pTC) feed with tetracycline at all time points.

TABLE 11
Comparison of the mice periodontal pocket depth (mm) in Lactobacillus salivarius
SG-M6 prevention groups (pLS) with day 4 at various time points and various concentration
(One-Way ANOVA and LSD multiple post hoc comparison; *p < 0.05; **p < 0.01).

| | pLSP1 | pLSP2 | pLSP4 | pLSP5 | pLSP10 | pLSP15 | pTC | pMT |
|---|---|---|---|---|---|---|---|---|
| Day 4 | 1.33 ± 0.58 | 0.50 ± 0.00 | 1.23 ± 0.54 | 0.90 ± 0.52 | 0.96 ± 0.50 | 0.60 ± 0.26 | 0.84 ± 0.50 | 2.22 ± 1.07 |
| Day 8 | 1.17 ± 0.76 | 0.83 ± 0.29 | 0.63 ± 0.75 | 0.59 ± 0.26 | 0.83 ± 0.50 | 0.91 ± 0.51* | 0.70 ± 0.34 | 2.16 ± 1.25 |
| Day 12 | 0.67 ± 0.29 | 1.67 ± 1.15 | 0.40 ± 0.17 | 0.50 ± 0.00 | 0.40 ± 0.15* | 0.40 ± 0.15** | 0.65 ± 0.26* | 2.75 ± 1.32 |
| Day 16 | 0.75 ± 0.35 | 1.50 ± 0.71 | 0.35 ± 0.21 | 0.20 ± 0.00 | 0.30 ± 0.17* | 0.40 ± 0.17* | 0.49 ± 0.25* | 2.21 ± 0.64 |

Inter group of various concentration Lactobacillus salivarius SG-M6 fermentation product at various time points was compared. Significant difference of loss of periodontal pocket depth was shown at day 12 and day 16 in pLSP10 and pTC group and at day 8, 12 and 16 in pLSP15 group.

### (2) Evaluation of Treating Effect for Animal Periodontal Disease

TABLE 12
Comparison of the mice periodontal pocket depth (mm) in Lactobacillus salivarius
SG-M6 treatment groups (tLS) against control group (tMT) at
different time points (One-Way ANOVA and Dunnett multiple post
hoc comparison; *p < 0.05; **p < 0.01)

| | Day 4 | Day 8 | Day 12 | Day 16 |
|---|---|---|---|---|
| tLSP1 | 1.83 ± 1.04 | 1.16 ± 1.15 | 1.50 ± 1.32 | 1.00 ± 0.00 |
| tLSP2 | 3.33 ± 1.53 | 1.50 ± 1.32 | 1.17 ± 1.15 | 0.83 ± 0.29 |
| tLSP4 | 3.33 ± 1.53 | 1.25 ± 1.06 | 0.65 ± 0.21** | 0.35 ± 0.21** |
| tLSP5 | 0.96 ± 0.47** | 0.75 ± 0.27** | 0.58 ± 0.40** | 0.20 ± 0.00** |
| tLSP10 | 1.21 ± 0.72** | 0.78 ± 0.26** | 0.61 ± 0.32** | 0.25 ± 0.23** |
| tLSP15 | 1.18 ± 0.72** | 0.88 ± 0.50** | 0.38 ± 0.16** | 0.20 ± 0.00** |
| tTC | 1.05 ± 0.64** | 0.89 ± 0.44** | 0.63 ± 0.37** | 0.48 ± 0.33** |
| tMT | 2.10 ± 0.63 | 1.91 ± 0.89 | 2.10 ± 0.97 | 2.00 ± 0.92 |

Effects of different concentrations of Lactobacillus salivarius SG-M6 fermentation product on periodontal pocket depth in treatment groups were evaluated. Significant differences of loss of periodontal pocket depth were shown from day 4 to day 12 and various concentrations, tLSP5, tLSP10 and tLSP15(p<0.05). Significant difference of improvement was shown in 4-fold concentration group (tLFP4) at day 12 and 16. Groups feeding with tetracycline (tCL) showed significant difference in all time points.

TABLE 13
Comparison of the mice periodontal pocket depth (mm) in Lactobacillus salivarius
SG-M6 treatment groups (tLF) with day 4 at various time points and various concentration
(One-Way ANOVA and LSD multiple post hoc comparison; *p < 0.05; **p < 0.01).

| | tLSP1 | tLSP2 | tLSP4 | tLSP5 | tLSP10 | tLSP15 | tTC | tMT |
|---|---|---|---|---|---|---|---|---|
| Day 4 | 1.83 ± 0.58 | 3.33 ± 1.53 | 3.33 ± 1.53 | 0.96 ± 0.47 | 1.21 ± 0.72 | 1.18 ± 0.72 | 1.05 ± 0.64 | 2.10 ± 0.63 |
| Day 8 | 1.16 ± 1.15 | 1.50 ± 1.32 | 1.25 ± 1.06 | 0.75 ± 0.27 | 0.78 ± 0.26 | 0.88 ± 0.50 | 0.89 ± 0.44 | 1.91 ± 0.89 |
| Day 12 | 1.50 ± 1.32 | 1.17 ± 1.15 | 0.65 ± 0.21 | 0.58 ± 0.40 | 0.61 ± 0.32** | 0.38 ± 0.16* | 0.63 ± 0.37** | 2.10 ± 0.97 |
| Day 16 | 1.00 ± 0.00 | 0.83 ± 0.29 | 0.35 ± 0.21 | 0.20 ± 0.00 | 0.25 ± 0.23** | 0.20 ± 0.00* | 0.61 ± 0.34** | 2.00 ± 0.92 |

Inter group comparison of periodontal pocket depth improvement in each treatment groups feeding with Lactobacillus salivarius SG-M6 fermentation product was compared with different time points (each group compared with Day 4). The periodontal pocket depth showed significant difference in 10-fold, 15-flod concentrate and tetracycline group, tLS P10, tLS P1 and tTC, at day 12 and 16.

### (3) Improvement Percentage of Periodontal Pocket Depth

TABLE 14
The periodontal pocket improvement percentage of Lactobacillus
salivarius SG-M6 prevention groups (pLS) showed significant difference
against no-treatment control group in prevention control (pMT) by
Mann-Whitney test (p < 0.05, percentage of improving individual).

| | Day 4 | Day 8 | Day 12 | Day 16 |
|---|---|---|---|---|
| pLSP1 | 0 | 33% | 67% | 100% |
| pLSP2 | 100% | 33% | 0 | 0 |
| pLSP4 | 67% | 67% | 33% | 100% |
| pLSP5 | 60% | 29% | 100% | 100% |
| pLSP10 | 42% | 44% | 50% | 100% |
| pLSP15 | 75% | 33% | 50% | 100% |
| pTC | 62% | 50% | 71% | 100% |
| pMT | — | — | — | — |

The periodontal pocket depth improvement percentage of Lactobacillus salivarius SG-M6 prevention groups all reached 100% at day 16 except for 2-fold concentrate groups.

TABLE 15
The periodontal pocket improvement percentage of Lactobacillus
salivarius SG-M6 treatment groups (tLS) showed significant
difference against no-treatment control group (tMT) by Mann-
Whitney test (p < 0.05, percentage of improving individual).

| | Day 4 | Day 8 | Day 12 | Day 16 |
|---|---|---|---|---|
| tLSP1 | 0 | 0 | 33% | 0 |
| tLSP2 | 0 | 33% | 67% | 33% |
| tLSP4 | 0 | 50% | 50% | 50% |
| tLSP5 | 36% | 38% | 40% | 50% |
| tLSP10 | 25% | 22% | 50% | 33% |
| tLSP15 | 45% | 38% | 60% | 50% |
| tTC | 30% | 20% | 47% | 33% |
| tMT | — | — | — | — |

Lactobacillus salivarius SG-M6 treatment groups all reached at least 33% improvement percentage at day 16 except for 1-fold group, tLSP1.

### (4) Clinical Pathological Analysis

The liver and kidney tissue section of the groups feeding Lactobacillus salivarius SG-M6 fermentation product (FIGS. 2, 3, 8 and 9) revealed no significant appearance of toxic effect and no inflammatory cell infiltration as compared to no-treatment control groups (tMT or pMT) (FIG. 4 to FIG. 7 and FIG. 10 to FIG. 13).

### CONCLUSION

In vitro study of disc agar diffusion test revealed that Lactobacillus salivarius SG-M6 fermentation product of more than 2-fold concentrate and live Lactobacillus salivarius formed inhibition zones against these four pathogens, Actinomyces viscosus, Porphyromonas gingivalis, Streptococcus mutans and Streptococcus sanguis. The higher the concentrated fold was presented, the larger the inhibition zone was produced which formed dose dependent manner. Broth dilution method test revealed that various concentrations of Lactobacillus salivarius SG-M6 fermentation product had different inhibition effects on periodontal pathogens. In general, higher concentration of Lactobacillus salivarius SG-M6 fermentation product had better inhibition effect. Co-culture data showed that Lactobacillus salivarius SG-M6 fermentation product had inhibition effect on these three periodontal pathogens, Porphyromonas gingivalis, Streptococcus mutans and Streptococcus sanguis in short period of time. The higher the concentration was used, the better the inhibition was obtained. The live Lactobacillus salivarius SG-M6 had inhibition effect on Streptococcus mutans. Live bacteria of 10⁷ cfu/mL Lactobacillus salivarius SG-M6 killed the Streptococcus mutans in 32 hours.

In vivo study of periodontal disease animal model showed that Lactobacillus salivarius SG-M6 fermentation product's preventive effect was better than its therapeutic effect on periodontal status improvement. For preventive effect, various feeding concentrations of Lactobacillus salivarius SG-M6 fermentation product showed significant improvement on periodontal pocket depth in all experiment groups, 15-fold, 10-fold, 5-fold, 4-fold, 2-fold and 1-fold concentrate. For therapeutic effect, fermentation product of 15-fold, 10-fold, 5-fold and 4-fold concentrate groups showed significant difference.

The mice in treatment groups feeding with Lactobacillus salivarius SG-M6 fermentation product were sacrificed at day 16 for pathological section of liver and kidney tissues. The results showed no significant appearance of toxic effect which represented its high safety.

In combination of in vitro and in vivo studies, it showed that salivarius SG-M6 fermentation product prevented the occurrence of periodontal disease and improved periodontal inflammation condition.

While the invention has been described and exemplified in sufficient detail for those skilled in this art to make and use it, various alternatives, modifications, and improvements should be apparent without departing from the spirit and scope of the invention. One skilled in the art readily appreciates that the present invention is well adapted to carry out the objects and obtain the ends and advantages mentioned, as well as those inherent therein. The animals, composition and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claim is:

1. A strain of Lactobacillus salivarius SG-M6 with deposit number of CGMCC NO. 3505.

2. The strain of claim 1, wherein said Lactobacillus salivarius SG-M6 includes nucleotide sequences shown in SEQ ID NO:1.

3. A fermentation product of Lactobacillus salivarius SG-M6, which is produced from fermentation broth of said Lactobacillus salivarius SG-M6 of claim 1.

4. The fermentation product of Lactobacillus salivarius SG-M6 of claim 3, wherein the fermentation product is produced after fermentation of medium culturing Lactobacillus salivarius SG-M6.

5. A health care composition for improving oral bacteria population, which comprises the Lactobacillus salivarius SG-M6 of claim 1.

6. The health care composition of claim 5, which is used for inhibiting the growth of oral periodontal bacteria in a subject.

7. The health care composition of claim 6, wherein the periodontal bacteria comprises Streptococcus mutans, Streptococcus sanguis, Porphyromonas gingivalis or Actinomyces viscosus.

8. The health care composition of claim 6, wherein the subject is mammals.

9. The health care composition of claim 8, wherein the subject is human.

10. The health care composition of claim 6, which is in the form of tablet, spray, solution, food or pharmaceuticals or oral hygiene product additives.

11. A health care composition for improving oral bacteria population, which comprises the fermentation product of claim 3.

12. The health care composition of claim 11, which is used for inhibiting the growth of oral periodontal bacteria in a subject.

13. The health care composition of claim 12, wherein the periodontal bacteria comprises Streptococcus mutans, Streptococcus sanguis, Porphyromonas gingivalis or Actinomyces viscosus.

14. The health care composition of claim 12, wherein the subject is mammals.

15. The health care composition of claim 14, wherein the subject is human.

16. The health care composition of claim 12, which is in the form of tablet, spray, solution, food or pharmaceuticals or oral hygiene product additives.
